(19) United States
(12) Plant Patent Application Publication
(10) Pub. No.: US 2015/0223383 P1
(43) Pub. Date: Aug. 6, 2015

(54) Grapevine "IASMA ECO 3"

(50) ;

(71) Applicant: FONDAZIONE EDMUND MACH-CENTRO RICERCA ED INNOVAZIONE, San Michele All'Adige (TN) (IT)

(72) Inventor: Marco STEFANINI, Bolzano (IT)

(73) Assignee: FONDAZIONE EDMUND MACH-CENTRO RICERCA ED INNOVAZIONE, San Michele All'Adige (TN) (IT)

(21) Appl. No.: 13/999,224

(22) Filed: Jan. 31, 2014

(51) Int. Cl. A01H 5/00

(52) U.S. Cl. PLT/207

(57) ABSTRACT

This invention is a new and distinct grapevine variety named “IASMA ECO 3” which is characterized by producing white berries which mature 1 week earlier than the parents, and is much less sensitive to Botrytis cinerea compared to the parents. The present variety berries exhibit a loose bunch and have the presence of both geraniol and linalool.

### LATIN NAME OF THE GENUS AND SPECIES CLAIMED

Vitis vinifera L.

### VARIETY DENOMINATION

“IASMA ECO 3”

### CROSS REFERENCE TO RELATED APPLICATIONS

The present application is related to Community Plant Variety application 2012/2344, filed in the Community Plant Variety Office on Oct. 25, 2012, the entire contents of which are hereby incorporated by reference.

### BACKGROUND OF THE INVENTION

### Discussion of the Background

The new and distinct grapevine described and claimed herein originated from a hand pollinated cross of Malvasia (Bianca) di Candia Aromatica (pollen parent) and Moscato Ottonel (seed parent), neither of which are protected by plant patent, plant variety protection certificate, or similar. The seeds were subsequently germinated and the resulting plants were planted in the field. The present variety of grapevine was selected as a single plant, and asexually propagated by hardwood cuttings. The resulting propagules were planted and were found to reproduce true-to-type through at least three generations of asexual reproduction. The observations took place from 2004 to 2010 in San Michele All'Adige (TN)—Trentino Alto Adige—Italy (46° 11′ Lat. N, 11° 28′ Long. E, 235 s.l.m.).

### SUMMARY OF THE INVENTION

To the inventor's knowledge, there are no varieties similar to the new variety; being a crossing result, they contain 50% of the genetic material of the seed parent and 50% of the genetic material of the pollen parent.

The present variety produces white berries like for the parents. The present variety matures 1 week earlier than the parents, and is much less sensitive to Botrytis cinerea compared to the parents. The present variety berries exhibit a loose bunch and have the presence of both geraniol and linalool.

### BRIEF DESCRIPTION OF THE FIGURES

FIG. 1 provides photographic illustration in full color of “IASMA ECO 3” in various views. The colors are as nearly true as is reasonably possible in a color representation of this type.

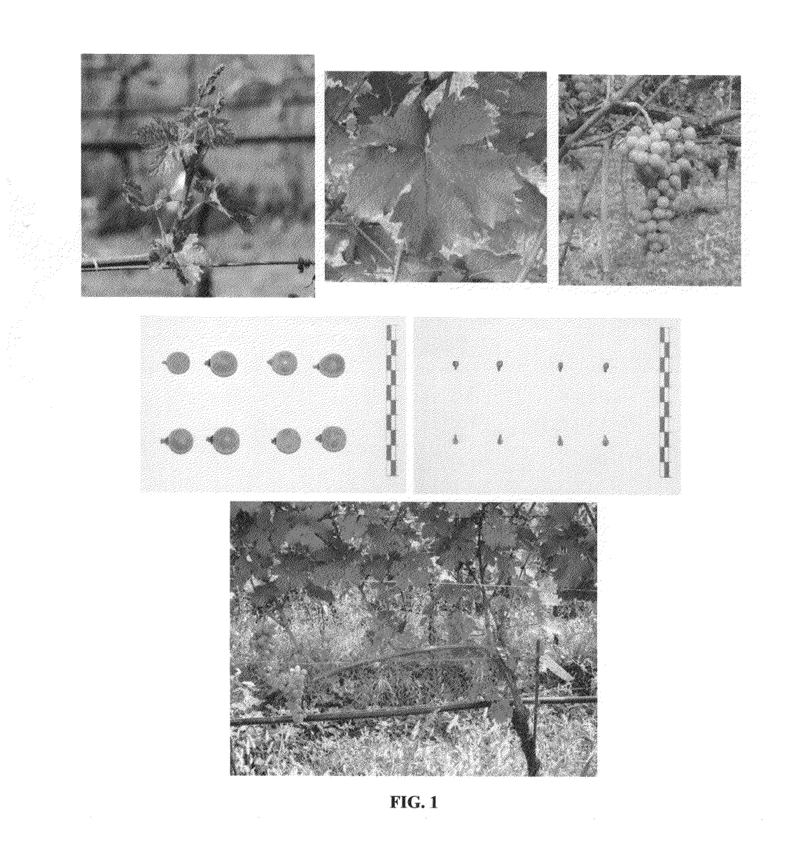

FIG. 2 provides a graphical representation of the average profile of aglycone flavonols in the present invention variety by percentile.

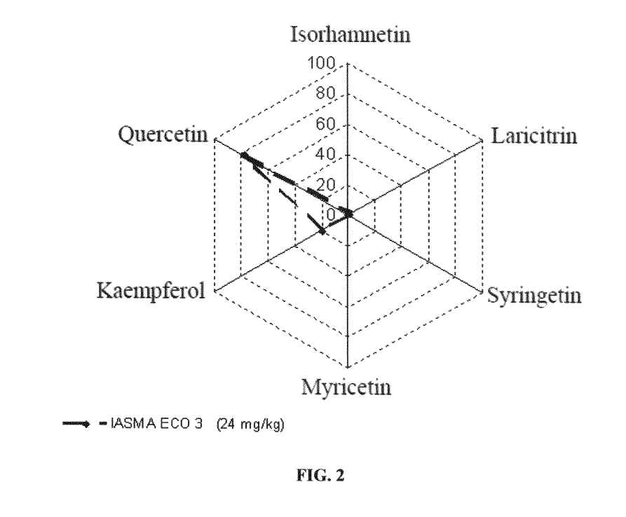

FIG. 3 provides a graphical representation of the average profile of cinnamic acids in the present invention variety by percentile and by mg/kg.

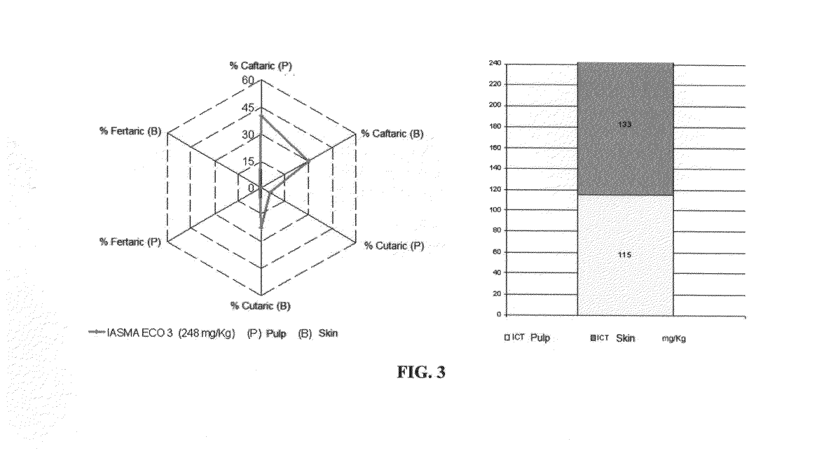

### DETAILED DESCRIPTION OF THE INVENTION

Throughout this specification, color names beginning with a small letter signify that the name of that color, as used in common speech, is aptly descriptive.

Throughout this specification subjective description values conform to those set forth by the Community Plant Variety Office (France) in the Technical Questionnaire for Grapevine varieties.

The descriptive matter which follows pertains to “IASMA ECO 3” plants grown in the vicinity of San Michele All'Adige (TN)—Trentino Alto Adige—Italy (46° 11′ Lat. N, 11° 28′ Long. E, 235 s.l.m.) from 2004 to 2010, and is believed to apply to plants of the variety grown under similar conditions of soil and climate elsewhere:
cod. OIV character:
Young shoot (10-30 cm): 001 aperture of tip 5 (fully open) 002 distribution of anthocyanin pigmentation of tip 1(absent) 003 intensity anthocyanin pigmentation of tip 1(none) 004 density prostrate hairs of tip 1 (none) 005 density erect hairs of extremity 1(none)
   Shoot on blooming: 006 attitude 3(semi-erect) 007 color of dorsal side of internodes 1(green) 008 color of ventral side of internodes 1(green) 009 color of dorsal side of nodes 1(green) 010 color of ventral side of nodes 1(green) 011 density erect hairs on nodes 1(none) 012 density erect hairs on internodes 1(none) 013 density prostrate hairs on nodes 1(none) 014 density prostrate hairs on internodes 1(none) 015-1 anthocyanin pigmentation on the latent bud scales 1 (absent) 015-2 intensity anthocyanin pigmentation bud scales 1 (none)
Tendrils: 016 number of consecutive tendrils 1(2 or less) 017 length 7 (long)
Young leaf: 051 color of the upper side 3 (bronze) 053 density prostrate hairs between the veins 1(none) 054 density erect hairs between the veins 1(none) 055 density prostrate hairs on veins 1(none) 056 density erect hairs on veins 1(none)
Mature leaf: 065 size of blade 3(small) 067 shape of blade 3 (pentagonal) 068 number of lobes 3 (five) 069 color of the upper side 3 (medium green) 070 anthocyanin pigmentation veins on the upper side 1(absent) 071 anthocyanin pigmentation veins on the lower side 1(absent) 072 goffering of blade 3 (weak) 073 undulation of blade between main and lateral veins 9 (present) 074 profile in cross section 4 (revolute) 075 blistering of upper side 3 (weak) 076 shape of teeth 3 (convex) 077 size of teeth in relation to blade size 5 (medium) 078 length of teeth compared with their width 7 (long) 079 degree of opening of petiole sinus 3 (little open) 080 shape of base of petiole sinus 3 (V-shaped) 081-1 teeth of the petiole sinus 1(absent) 081-2 petiole sinus base delimited by veins 1(absent) 082 degree of opening of upper lateral sinuses 3 (slightly overlapped) 083-1 shape of base of upper lateral sinuses 2 ({-shaped) 083-2 teeth in the upper lateral sinuses 1 (absent) 084 density of prostrate hairs between the veins of the lower side 1 (absent) 085 density of erect hairs between the veins of the lower side 1(absent) 086 density of prostrate hairs on the veins of the side 1 (none) 087 density of erect hairs on the veins of the lower side 7 (high) 088 prostrate hairs on the main veins of the upper side 1(absent) 089 erect hairs on the main veins of the upper side 1(absent) 090 density of prostrate hairs on petiole 1(none) 091 density of erect hairs on petiole 1 (none) 093 length of petiole compared to middle vein 3 (slightly shorter)
Woody shoot: 101 cross section 2 (elliptic) 102 structure of surface 3 (striate) 103 main color 2 (brownish)
Inflorescence 151 sexual organs 3 (stamens complete, gynoecium developed)
Bunch: 202 length 5 (medium,<16 cm) 203 width 3(narrow,<8cm) 204 density 5 (medium) 206 length of peduncle 5 (medium) 207 lignification of peduncle 1(at the base only) 208 shape 2 (conical) 209 number of wings 1 (absent)
Berry: 220 length 5 (medium,<18mm) 221 width 5 (medium,<18 mm) 222 uniformity of size 9 (uniform) 223 shape 2 (globose) 224 color of the skin 1 (green yellow) 226 uniformity of the color of the skin 2 (uniform) 227 bloom 5 (medium) 228 thickness of the skin 5 (medium) 229 hilum 1 (little visible) 230 intensity anthocyanin pigmentation of the flesh 1(none) 232 juiciness of the flesh 2 (medium juicy) 233 must yield 3 (low) 235 firmness of the flesh 2 (slightly firm) 236 particular flavor 2 (muscat) 238 length of pedicel 3 (short,<7 mm) 240 ease of detachment from pedicel 5 (medium) 241 formation of seeds 2 (complete) 306 time of autumn coloring of leaves 1 (yellow)
Phenologic characteristics: Time of bud burst: medium Time of full bloom: medium Time of berry ripening: medium Time of maturity: medium
Productive characteristics: Real fertility of the buds 1.45 Potential fertility 1.62 Position of the first bunch 2nd-3rd bud Production of grapes per vine Kg 2.25 Yield per hectare quintal 96 Medium weight bunch g 189 Medium weight berry g 2.10 Sugars, Brix degree 22.52 Total acid content g/15.82 pH 3.46

The present variety tested negative against each of the GFLV, ArMV, GLRaV-1, GLRaV-3, and GVA viruses.

### FINDINGS OF MOLECULAR INVESTIGATIONS

DNA was extracted from young leaves of different accessions for each variety by using the Doyle and Doyle extraction method (1990) as modified by Grando et al. (2003). Molecular markers were obtained by analyzing DNA specimens at the 9 microsatellite loci specified by the European projects named Gen-Res81 and Grape-Gen06 for vine variety characterisation and at locus VMC1B11 (Vitis Microsatellite Consortium). In particular, the primers and amplification conditions described by Thomas and Scott (1993) for locus VVS2, Bowers et al. (1996) for loci VVMD5 and VVMD7, Bowers et al. (1999) for loci VVMD25, VVMD27, VVMD28 and VVMD32 and Sefc et al. (1999) for loci VrZAG62 and VrZAG79 were applied. PCR products were separated and analyzed with the Genetic Analyzer ABI3130 and the GeneMapper® v4.0 software.

| | Microsatellite markers | | | | | | | | | | | | | | | | | | | |
|---|---|---|---|---|---|---|---|---|---|---|---|---|---|---|---|---|---|---|---|---|
| IASMA | WMD5 | | WMD7 | | VVMD25 | | VMD27 | | VVMD28 | | WMD32 | | VVS2 | | VMC1B11 | | VrZAG62 | | VrZAG79 | |
| ECO 3 | 225 | 227 | 233 | 239 | 250 | 256 | 177 | 187 | 249 | 259 | 241 | 265 | 130 | 132 | 185 | 185 | 193 | 203 | 255 | 259 |

### FINDINGS OF CULTIVATION TESTS

The series of summary tables below contain the findings for various vintage years concerning the “IASMA ECO 3” vine variety compared to the reference vine variety.

Average data relative to some plant production parameters observed at the San Michele a/A (TN) vineyard (vintage years 2004-2010): vine grown following the Guyot method (arched shoot pruned at 8-10 sprouts/vine).

| | IASMA ECO 3 | CHARDONNAY |
|---|---|---|
| | S. Michele a/A | S. Michele a/A |
| Grape/vine production (kg) | 2.25 | 3.22 |
| Actual shoot fertility | 1.45 | 1.49 |
| Potential shoot fertility | 1.62 | 1.54 |
| Wood/vine production (kg) | 0.489 | 0.686 |
| Must sugars (° Brix) | 22.52 | 20.06 |
| Moulds (%) | 5.3 | 7.5 |
| bunch botrytis | | |
| Moulds (%) | 0.00 | 0.0 |
| rachis desiccation | | |
| Grape/wood ratio | 4.60 | 4.69 |

Average data relative to mechanical bunch analysis and must chemical composition. Collection of bunches in the vintage years 2004-2010 at technological maturity (first or second half of September). Vineyard in S. Michele a./A. (Girelli TN).

| | Values | | |
|---|---|---|---|
| | | Limit v. | |
| IASMA ECO 3 | Average v. | Min | Max |

Mech. bunch analysis

| | | | | |
|---|---|---|---|---|
| bunch average | g | 189 | 158 | 323 |
| weight | | | | |
| berry average | g | 2.10 | 1.85 | 2.60 |
| weight | | | | |
| berry longitudinal | nun | 18 | 14 | 22 |
| diameter | | | | |
| berry average | nun | 17 | 12 | 19 |
| diameter | | | | |
| Bunch | | | | |
| composition | | | | |
| berries | (% in weight) | 95.3 | 94.1 | 96.6 |
| stems | (% in weight) | 4.7 | 3.4 | 5.9 |

Berry composition

| | | | | |
|---|---|---|---|---|
| skins | (% in weight) | 23.8 | 18.5 | 27.6 |
| seeds | (% in weight) | 4.2 | 2.9 | 5.8 |
| pulp and must | (% in weight) | 72.0 | 63.5 | 77.3 |
| must yield | (% volume/weight) | 68.4 | 60.5 | 71.1 |

Must chemical composition

| | | | | |
|---|---|---|---|---|
| sugars | ° Brix | 22. | 20.00 | 25.20 |
| | | 52 | | |
| total acidity FA | | 5.82 | 4.43 | 6.98 |
| pH | | 3.46 | 3.25 | 3.95 |
| tartaric acid | g/l | 5.47 | 3.94 | 7.02 |
| malic acid | g/l | 2.08 | 1.44 | 3.02 |
| potassium | g/l | 1.46 | 1.11 | 2.32 |

Average data relative to the analysis of secondary metabolite profiles (aglycone flavonols, cinnamic acids and aromatic compounds of fruits) that characterise the variety's compositional phenotype.

Average profile of aromatic compounds in the free and complexed form

AROMATIC COMPOUNDS

| | Oxide | Oxide | | 4- | HO- | Oxide | Oxide | citro- |
|---|---|---|---|---|---|---|---|---|
| | A | B | linalol | terpineol | trienol | C | D | nellol |
| IASMA ECO | 16 | 15 | 138 | <1 | 74 | 12 | 85 | 22 |
| 3 (free) | | | | | | | | |
| IASMA ECO | 264 | 58 | 473 | <1 | 109 | 53 | 52 | 14 |
| 3 (complexed) | | | | | | | | |
| | Methyl | | gera- | benzyl | b-phenylethyl | HO- | HO- | hydroxy- |
| | salicylate | nerol | niol | alcohol | alcohol | diol I | diol II | citronellol |
| IASMA ECO | 1 | <1 | 14 | 117 | 24 | 14 | 68 | 246 |
| 3 (free) | | | | | | | | |
| IASMA ECO | 53 | <1 | 186 | 557 | 102 | 118 | 18 | 24 |
| 3 (complexed) | | | | | | | | |
| | hydroxy- | 8-hydroxy- | 8-hydroxy- | 7-hydroxy- | Trans-geranic | | rose | rose |
| | nerol | linalol trans | linalol cls | geraniol | acid | phenol | oxide I | oxide II |
| IASMA ECO | <1 | 15 | 25 | 18 | 2 | 29 | <1 | <1 |
| 3 (free) | | | | | | | | |
| IASMA ECO | <1 | 232 | 171 | 133 | 30 | 906 | <1 | <1 |
| 3 (complexed) | | | | | | | | |
| | | | benzal- | 3-hoxy- | 3-hydroxy- | | 3-hexanol | 3-hexanol |
| | | | dehyde | 0-ionol | damascone | hexanol | trans | cis |
| | | IASMA ECO | <1 | 10 | <1 | 88 | <1 | 47 |
| | | 3 (free) | | | | | | |
| | | IASMA ECO | <1 | 78 | <1 | 12 | <1 | 16 |
| | | 3 (complexed) | | | | | | |

Values expressed as μg/Kg of n-heptanol standard grape RF = 1

According to the average profile of aromatic compounds in the free and complexed form, this variety can be classified as an aromatic variety.

Wine analysis.
Origin of grape: Vineyard in S. Michele a./A. (Girelli TN)
vintage years 2004-2010.

| | Values | | |
|---|---|---|---|
| | | Limit v. | |
| IASMA ECO 3 | Average v. | Min. | Max |
| alcohol content % vol. | 12.81 | 11.12 | 13.43 |
| total acidity in tartaric acid g/l | 4.91 | 3.75 | 5.15 |
| dry extract g/l | 20.60 | 17.38 | 22.50 |
| pH | 3.50 | 3.45 | 3.65 |
| catechins and proantocyanidins mg/l | 20 | 14 | 29 |
| (+) catechin | | | |
| total polypheuols mg/l (+) catechin | 212 | 148 | 264 |

### BEHAVIOUR IN VEGETATIVE MULTIPLICATION

Grafted vine affinity with the most popular holders—3) good

### CULTIVATION CHARACTERISTICS AND SUITABILITY

The present invention vine variety shows good vigour with medium sprouting stage, with green-yellow, medium-sized, spheroid berry with medium thickness skin. The bunch is medium-sized, conical, relatively compact and often winged. It has a good yield and is relatively quite resistant to botrytis. Its physiological maturity occurs in the second stage; it shows a good capacity to accumulate sugars with low acidity values, especially in warm vintage years. It can perfectly stand winter cold and shows a good resistance to downy mildew and a medium resistance to powdery mildew. It prefers relatively fertile mixed hilly soils. It is ideal to produce delicate aromatic wines at altitudes exceeding 400 m above sea level or on slopes facing north.

### USE

The present invention vine variety is particularly suited to produce fresh aromatic white wine; its taste recalls lemon balm and white peach with intense organoleptic characteristics; medium-low acidic strength (low in warm vintage years). It produces medium-body wines with good alcohol content and good sapidity, slightly aromatic and very peculiar. It can also be successfully used to produce still pure or mixed wines.

### REFERENCES

BOWERS J. E., DANGL G. S., VIGNANI R., MEREDITH C. P. (1996)—Isolation and characterization of new polymorphic simple sequence repeat loci in grape (Vitis vinifera L.). Genome 39, 628-633.
BOWERS, J. E.; DANGL, G. S.; MEREDITH, C. P.; 1999b: Development and characterization of additional microsatellite DNA markers for grape. Am. J. Enol. Vitic. 50 (3), 243-246.
Costantini L., Monaco A., Vouillamoz J. F., Forlani M., Grando M. S. (2005) Genetic relationships among local Vitis vinifera cultivars from Campania (Italy) Vitis 44 (1) 25-34
This P, Jung A, Boccacci P, Borrego J, Botta R, Costantini L, Crespan M, Dangl G. S, Eisenheld C, Ferreira-Monteiro F., Grando MS., Ibáñez J, Lacombe T, Laucou V, Magalhaes R, Meredith C. P, Milani N, Peterlunger E, Regner F, Zulini L, Maul E (2004). Development of a standard set of microsatellite reference alleles for identification of grape cultivars. Theor Appl Genet 109: 1448-1458
SEFC, K. M.; REGNER, F.; TURETSCHEK, E.; GLÖSSL, J.; STEINKELLNER, H.; 1999: Identification of microsatellite sequences in Vitis riparia and their applicability for genotyping of different Vitis species. Genome 42, 367-373.
SOLDAVINI C., SCHNEIDER A., STEFANINI M., DALLASERRA A., POLICARPO M. (2007). SuperAmpelo un Software per la descrizione ampelografica e ampelometrica della vite Italus Hortus 14, 2: 39-40.
STEFANINI M., GRANDO M. S., IACONO F. (1998) Recupero, caratterizzazione e valorizzazione di vecchie variety di Vitis vinifera coltivate in Trentino. Atti Biodiversity: germoplasma locale e sua valorizzazione pp. 269-272. (Alghero)
STEFANINI M. TOMASI T. (2010) Antichi vitigni del Trentino FEM Istituto Agrario S. Michele a/A (TN)
THOMAS M. R., SCOTT N. S. (1993)—Microsatellite repeats in grapevine reveal DNA polymorphisms when analysed as sequence-tagged sites (STSs). Theor. Appl. Genet. 86, 985-990.
RAUZI G. M. SPAGNOLLI F. Note storico-etimologiche sulla viticoltura trentina XIV Congresso internazionale della vite e del vino Trento (1974)
MARCHI C. Sul nuovo indirizzo viticolo-enologico nella Venezia Tridentina. (1930)

1. A new and distinct variety of grapevine as herein illustrated and described.
